(19) United States
(12) Patent Application Publication
(10) Pub. No.: US 2023/0047316 A1
(43) Pub. Date: Feb. 16, 2023

(54) AUTONOMOUSLY NAVIGATING ROBOT CAPABLE OF CONVERSING AND SCANNING BODY TEMPERATURE TO HELP SCREEN FOR COVID-19 AND OPERATION SYSTEM THEREOF

(71) Applicant: Ryan H. KIM, Seoul (KR)

(72) Inventor: Ryan H. KIM, Seoul (KR)

(21) Appl. No.: 17/573,474

(22) Filed: Jan. 11, 2022

(30) Foreign Application Priority Data
Aug. 10, 2021 (KR) 10-2021-0105412

(51) Int. Cl. B25J 9/16; A61B 5/00; B25J 19/02; A61B 5/01

(52) U.S. Cl. CPC B25J 9/1664; A61B 5/6898; B25J 19/023; A61B 5/0077; B25J 9/161; B25J 9/1689; A61B 5/01; B25J 9/162

(57) ABSTRACT

This application relates to an autonomously navigating robot. In one aspect, the robot includes an end effector configured to measure a person's body temperature and, when the body temperature exceeds a standard fever temperature, activate a chatbot to check symptoms of Covid-19. The robot may also include a manipulator configured to align the end effector with the person's forehead. The robot may further include a mobile robot configured to detect the person and move the end effector and the manipulator to a position where the person is located by performing autonomous navigation.

### CROSS-REFERENCE TO RELATED APPLICATION

This application is based on and claims priority under 35 U.S.C. § 119 to Korean Patent Application No. 10-2021-0105412, filed on Aug. 10, 2021, in the Korean Intellectual Property Office, the disclosure of which is incorporated by reference herein in its entirety.

### BACKGROUND

### Technical Field

The present disclosure relates to an autonomously navigating robot capable of having a conversation and measuring a body temperature from the distance with a thermal camera to help screen for COVID-19, and an operation system thereof.

### Description of Related Technology

During the coronavirus (COVID-19) pandemic of 2019, the most common symptom of COVID-19 patients has been fever. Therefore, countries and organizations around the world check the body temperature of people as a preemptive measure of detecting potential carriers of the virus.

In general, a process of measuring the body temperature is performed in such a way that a facility manager measures the body temperatures of facility entrants with a portable thermometer. In addition, some countries and organizations use kiosks to measure the temperature, but these two methods of measuring the temperature have many problems.

### SUMMARY

The present disclosure provides an autonomously navigating robot capable of conveniently testing a person for COVID-19 by measuring from the distance a body temperature of a person passing by and asking and answering questions of the person, and an operation system thereof.

Additional aspects will be set forth in part in the description which follows and, in part, will be apparent from the description, or may be learned by practice of the presented embodiments of the disclosure.

An autonomously navigating robot according to an embodiment includes an end effector that measures a person's body temperature by using a thermal camera from the distance and, when the body temperature exceeds a certain temperature, activates an alarm and activates a chatbot to check questions and answers related to symptoms of COVID-19, a manipulator configured to align the end effector with the person's forehead, and a mobile robot configured to detect the person and move the end effector and the manipulator to a position where the person is located by performing autonomous navigation.

In the autonomously navigating robot according to the embodiment, the mobile robot may include a personal computer for controlling movement of the mobile robot.

In the autonomously navigating robot according to the embodiment, the mobile robot may include a lidar system that detects a surrounding environment to perform simultaneous localization and mapping (SLAM), autonomous search, and path planning.

In the autonomously navigating robot according to the embodiment, the manipulator may be installed above the lidar system.

In the autonomously navigating robot according to the embodiment, the manipulator may include three actuators.

In the autonomously navigating robot according to the embodiment, one actuator among the three actuators may be used to perform a yaw motion, and the other two actuators are used to perform a pitch motion.

In the autonomously navigating robot according to the embodiment, the end effector may include a Universal Serial Bus (USB) camera that provides a real-time image.

In the autonomously navigating robot according to the embodiment, the real-time image provided by the USB camera may be transmitted to a personal computer of the mobile robot, and the person may be detected via a you-only-look-once (YOLO) algorithm executed by the personal computer.

In the autonomously navigating robot according to the embodiment, when the person is detected, the YOLO algorithm may generate a rectangular bounding box centered on the person's face, calculate coordinates therefor, and generate an actuator command for the manipulator through coordinate information.

In the autonomously navigating robot according to the embodiment, the YOLO algorithm may acquire a coordinate value of the person's face and acquires a temperature value of a point of the coordinate value.

In the autonomously navigating robot according to the embodiment, the end effector may include a thermal camera for determining the person's body temperature.

In the autonomously navigating robot according to the embodiment, one end of a fixed portion and one end of a thermal camera fixed hanger may be arranged between the thermal camera and the USB camera.

In the autonomously navigating robot according to the embodiment, the end effector may include a fixed portion for forming a skeleton of the end effector, and a thermal camera fixed hanger for fixing the thermal camera arranged in the end effector.

In the autonomously navigating robot according to the embodiment, a USB camera, the thermal imager, and a phone case may be mounted on the fixed portion.

In the autonomously navigating robot according to the embodiment, the end effector may be equipped with a smartphone that provides a user interface.

In the autonomously navigating robot according to the embodiment, an Android custom application of the smartphone may display, on a thermal image provided by the thermal camera, a point with a highest temperature among nine temperature points including one temperature point of provided coordinates and eight temperature points around the provided coordinates.

In the autonomously navigating robot according to the embodiment, when a temperature exceeding a fever threshold value among the nine temperature points is detected, a screen of the smart phone may be changed to a help screen, and a natural language understanding artificial intelligence chatbot may be activated.

In the autonomously navigating robot according to the embodiment, the natural language understanding artificial intelligence chatbot may converse with a user about vaccinations and potential symptoms of Covid-19, and when a predefined set of intents and 10 to 15 sample phrases for each intent are provided, a framework of a system of a chatbot may respond by accurately extracting a meaning of each question asked by a person and learning itself to match the intents.

An autonomously navigating robot operation system according to an embodiment includes instructions for performing autonomous navigation by using an autonomously navigating robot, detecting a person by using the autonomously navigating robot, aligning an end effector with the person's forehead by stopping the autonomously navigating robot and moving a manipulator, and checking the person's body temperature and, when the body temperature exceeds a standard fever temperature, activating a chatbot to converse with the person and check the person for symptoms of COVID-19.

A program according to an embodiment may be stored in a medium for a computer to perform an autonomously navigating robot operation system.

Other aspects, features and advantages other than those described above will become apparent from the following detailed description, claims and drawings for implementing the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a view illustrating an autonomously navigating robot according to an embodiment of the present disclosure.

FIG. 2 is a view illustrating a shape of an end effector according to an embodiment of the present disclosure.

FIG. 3 is a flowchart illustrating a chatbot function of an end effector according to an embodiment of the present disclosure.

FIG. 4 is a view illustrating a shape of a manipulator according to an embodiment of the present disclosure.

FIG. 5 is a diagram illustrating a shape in which an end effector according to an embodiment of the present disclosure is aligned in a rectangular bounding box drawn around a person's face.

FIG. 6 is a view illustrating coordinate axes of a manipulator according to an embodiment of the present disclosure.

FIG. 7 is a shape of FIG. 6 viewed from an upper side.

FIG. 8 is a shape of FIG. 6 viewed from a side.

FIG. 9 is a view illustrating a shape of a mobile robot according to an embodiment of the present disclosure.

FIG. 10 is a view illustrating internal configurations of a mobile robot according to an embodiment of the present disclosure.

FIG. 11 is a flowchart illustrating a running system of an autonomously navigating robot according to an embodiment of the present disclosure.

### DETAILED DESCRIPTION

One of the problems is safety of a facility manager who measures the body temperature. A person measuring the body temperature measures the temperature of the forehead of a person entering a facility with a portable thermometer, and in this case, there is no choice but to measure the temperature while the person measuring the temperature and a person whose temperature is being measured are located adjacent to each other. Thus, it is difficult to keep the distance between the person measuring the temperature and the person whose temperature is being measured.

In addition, when a person's temperature is measured by using a kiosk, the person has to directly position his/her face on an infrared camera of the kiosk that is fixed. In this case, it is difficult to properly align the face of the person whose temperature is being measured with the infrared camera, and thus, the person whose temperature is being measured feels uncomfortable, time is wasted, and sometimes even the body temperature is measured incorrectly.

In addition, when a person's temperature is measured by using a kiosk, the kiosk measures the temperature while being fixed in one place, and thus, in order to systematically measure the temperatures of many persons, entry points need to be limited. When the entrance is limited in this way, a bottleneck due to many persons subject to temperature measurement and a long waiting time may occur, which may cause inconvenience and waste of time for the many persons whose temperature is to be measured.

In addition, use of fixed devices (immobility) means that multiple kiosks are required to cover large areas. In a case where the kiosk is located outdoors, when an outdoor temperature is low such as in winter, a temperature of the face may be reduced temporarily due to an outdoor temperature less than the body temperature, and thus, the body temperature of a person having fever may be incorrectly measured as being normal.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The present disclosure may be transformed in various forms and have various embodiments, and some embodiments are illustrated in the drawings and described in detail in the specification of the present disclosure. However, this is not intended to limit the present disclosure to some embodiments, and the present disclosure should be understood to include all modifications, equivalents and substitutes included in the idea and scope of the present disclosure. In describing the present disclosure, the same numbers are used for the same components although illustrated in other embodiments.

Hereinafter, embodiments of the present disclosure will be described in detail with reference to the accompanying drawings, and when described with reference to the drawings, the same or corresponding components are given the same reference numerals, and overlapping descriptions thereof are omitted.

In the following embodiments, terms such as first, second, and so on are used for the purpose of distinguishing one component from another without being used in a limiting sense.

In the following embodiments, a singular expression includes a plural expression unless the context clearly dictates otherwise.

In the following embodiments, terms such as include and have indicate that features or components described in the specification are present, and probability that one or more other features or components may be added is not excluded in advance.

In the drawings, sizes of components may be exaggerated or reduced for the sake of convenient description. For example, a size and a thickness of each component in the drawings are randomly illustrated for the sake of convenient description, and the present disclosure is not limited to the illustration.

In the following embodiments, an x axis, a y axis, and a z axis are not limited to three axes on an orthogonal coordinate system and may be interpreted in a broader sense including the axes. For example, the x axis, the y axis, and the z axis may be orthogonal to each other but may refer to different directions that are not orthogonal to each other.

When certain embodiments are otherwise practicable, a certain process sequence may also be performed differently from the described sequence. For example, two processes described in succession may also be performed substantially simultaneously or may also be performed in an order opposite to the described order.

The terms used in the present application are only used to describe some embodiments and are not intended to limit the present disclosure. In the present application, terms such as “include” or “have” are intended to designate that features, numbers, steps, operation, configuration elements, components, or combinations thereof described in the specification exist, and it should be understood that probability of addition or existence of one or more other features, numbers, steps, operations, configuration elements, components, or combinations thereof is not precluded.

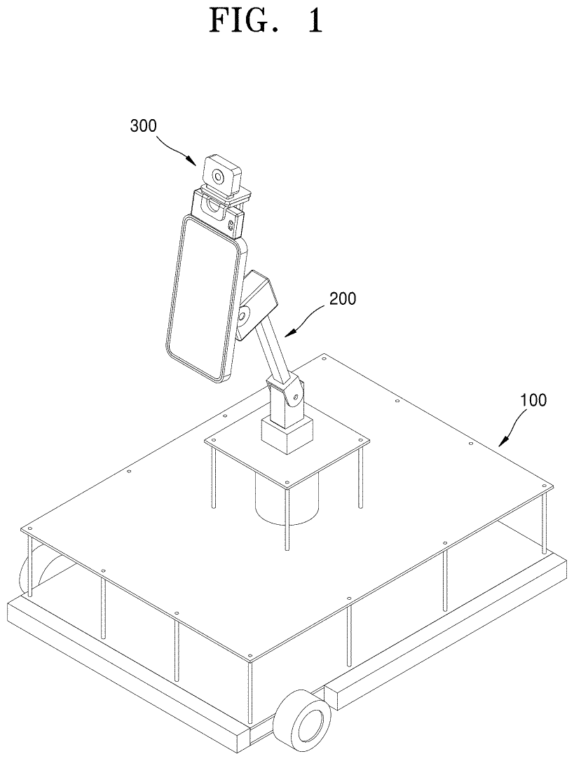

FIG. 1 is a view illustrating an autonomously navigating robot according to an embodiment of the present disclosure.

The autonomously navigating robot according to the embodiment of the present disclosure illustrated in FIG. 1 may autonomously perform navigation and path search. In addition, the autonomously navigating robot may accurately find and follow a person's face. In addition, the autonomously navigating robot may measure a temperature of a person's face and converse with a user about symptoms of Covid-19.

The operations described above may be performed by an end effector 300, a manipulator 200, and a mobile robot 100. In this case, a sub-module of the end effector 200 may include a custom application and a thermal camera including a programmable software development kit (SDK) to extract a person's body temperature.

If the temperature exceeds a fever threshold value of 38° C./100.4° F. defined by the centers for disease control and prevention (CDC), a natural language understanding (NLU) artificial intelligence (AI) chatbot that converses with a user about symptoms of COVID-19 may be activated.

According to the present embodiment, the chatbot may be activated only when a temperature is measured three times and the measured temperatures exceed the fever threshold value. This is because a thermal camera may inaccurately measure a temperature or may be affected by a surrounding object, weather, a surrounding environment, and so on, and thus, the temperature is measured three times, and only when all the measured temperature values exceed the fever threshold value, the chatbot may be activated to increase accuracy of temperature measurement.

The manipulator 200 may detect a person by using an object-recognition algorithm, and a custom inverse kinematics algorithm is used to orient the manipulator 200 and the end effector 300 to align with a detected person's forehead.

The mobile robot 100 may be used for autonomous navigation and path planning. The autonomously navigating robot may detect and avoid an obstacle and may stop when a person is detected.

An Android operating system may be used to operate the end effector 300. In this case, a robot operating system (ROS) may be used to operate the manipulator 200 and a mobile base. The autonomously navigating robot according to the present embodiment may be driven by using Android 10 as the Android operating system and ROS 1 Melodic Morenia as the ROS.

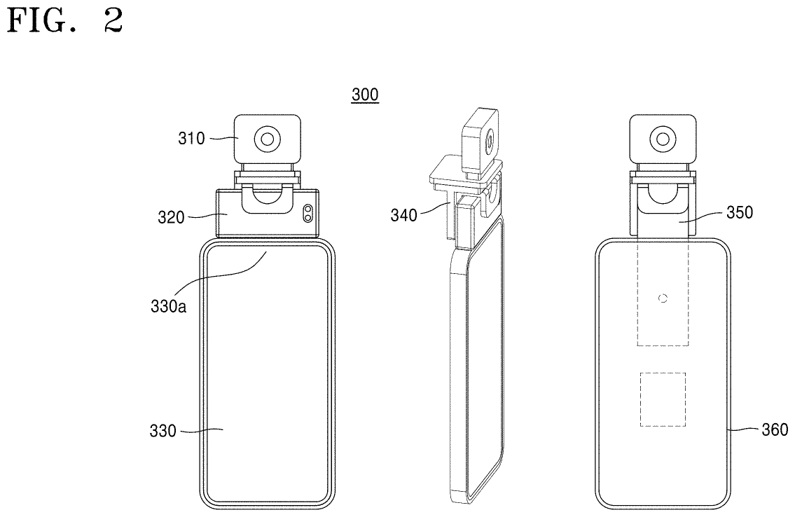

FIG. 2 is a view illustrating a shape of the end effector 300 according to the embodiment of the present disclosure.

Referring to FIGS. 1 and 2, the end effector 300 attached to the manipulator 200 may be used to scan a person's body temperature and to communicate with a person with a fever when the person has a fever. According to an embodiment of the present disclosure, the end effector 300 may include four sub-modules.

The thermal camera 320 may be connected to a smartphone by using a Universal Serial Bus type C (USB-C) port and may provide an accurate temperature value when coordinates thereof are given.

A USB camera 310 attached to the end effector 300 may provide a real-time image for object recognition, and thereby, the manipulator 200 may find a person. In particular, the USB camera 310 may recognize a person.

An Android custom application according to an embodiment of the present disclosure may perform an interaction with a user interface (UI), an interaction with the thermal camera 320 (Interaction), and an interaction with a chatbot.

The UI according to the present embodiment may be used to display temperature data for a user. Extending a view class, a custom camera view may be placed in the center of a screen to display a view from a front facing camera. In this case, a marquee text may be placed above the camera view and cycles phrases “Hello I am a mobile temperature scanner. I will be measuring your temperature and checking for a fever. Please wear your mask and socially distance. Thank you.” may be displayed repeatably.

The phrases may be verbally repeated every ten seconds by using a timer. Through the marquee text and text-to-speech (TTS) engine, it is possible to prevent suspicion or distrust of people around the robot by notifying the intention of the robot. The marquee text and a text-to-speech (TTS) engine inform a person around the robot of intent of a robot to prevent suspicion or mistrust of the person.

In an Android custom app, a first application may provide face detection, and a second application may receive data and draw a bounding box around each face. A thermal image of the thermal camera 320 may provide its own image with temperature values, on which coordinates and a bounding box are mapped.

In this case, temperature values from the provided coordinates and eight points around the coordinates may be collated with each other, and a point with the highest temperature among the nine points on the face may be displayed. This is because, when the manipulator 200 vibrates due to movement or when a person passes by the autonomously navigating robot too quickly, it is difficult to accurately detect a person's body temperature.

Thereafter, when a temperature exceeding the fever threshold value of 38° C./100.4° F. defined by the CDC is detected, a screen is changed to a help screen, and the NLU AI chatbot may be activated. The chatbot may converse with a user about vaccinations and potential symptoms of COVID-19.

When intents of conversation for conversation with a body temperature measurement target person are predefined and when 10 to 15 sample phrases are provided for each intent, a developed chatbot framework trains itself to accurately extract a meaning of each of the phrases and match the phrases to the intents. In this case, machine learning and natural language understanding may be used to analyze meaning of a user's input.

When the meaning extraction through the framework matches predefined intents, the chatbot may respond with predefined responses. If not, an operation system may respond with phrases like “I'm sorry I didn't understand what you said” and prompt a user to repeat a command.

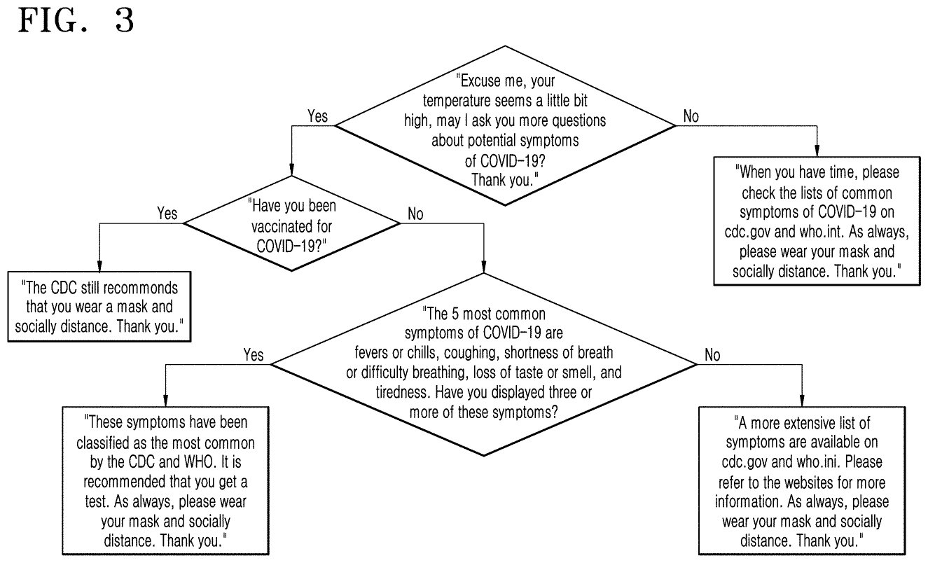

The chatbot may receive an input by an on-screen keyboard or through a speech-to-text (STT) engine. The chatbot may respond verbally by using text displayed on a screen and a TTS engine. A conversation flow of the chatbot is illustrated in FIG. 3.

Referring to FIG. 2, for physical coupling of sub-modules, a smartphone 330 is placed in an inverted state in a phone case 360 to reduce a distance between the thermal camera 320 and the USB camera 310 to reduce parallax. This is because the thermal camera 320 may be coupled to a lower portion 330a of the smartphone 330.

The smartphone 330 is held in place by the phone case 360 which is screwed onto the manipulator 200. A thermal camera fixed hanger 350 may fix the thermal camera 320 between the phone case 360 and the USB camera 310. A fixed portion 340 may form a skeleton of the end effector 300, the USB camera 310 may be mounted on an upper end of the fixed portion 340, the thermal camera 320 may be mounted on the middle of the fixed portion 340, and the phone case 360 may be mounted on a lower end of the fixed portion 340. The thermal camera fixed hanger 350 may be mounted on the fixed portion 340.

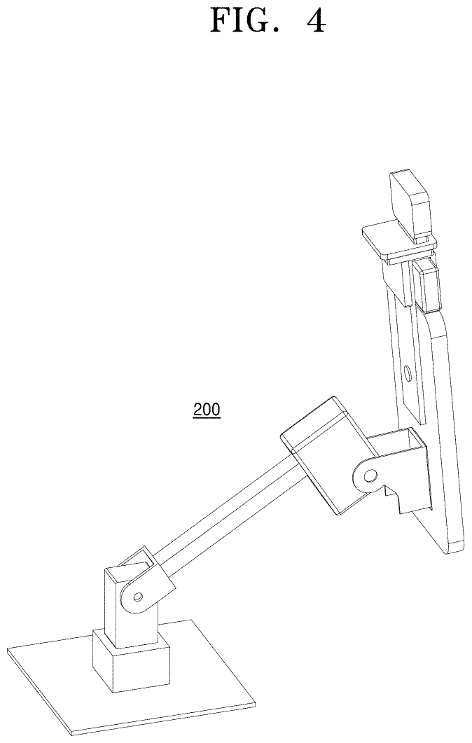

FIG. 4 is a view illustrating a shape of a manipulator according to an embodiment of the present disclosure.

The manipulator 200 may be used to detect and follow a person such that the end effector 300 is aligned with the forehead.

The manipulator 200 may be used to detect and follow a person to orient the end effector 300 to align with the person's forehead. In order to detect a person, the USB camera 320 attached to the end effector 300 may be used to send real-time images to a computer mounted on the mobile robot 100. For tracking a person, a detection algorithm that is both fast and accurate may be required.

To this end, according to the present embodiment, a person may be detected via a you-only-look-once (YOLO) algorithm. The YOLO algorithm may detect images with accuracy comparable to other convolutional neural networks (CNNs) but at faster speeds. The YOLO algorithm may be executed by a personal computer (PC) 110 of the mobile robot 100.

Upon detecting a person via the YOLO algorithm, the mobile robot 100 may be programmed to temporarily stop. Through this, the end effector 300 may more stably measure a person's body temperature.

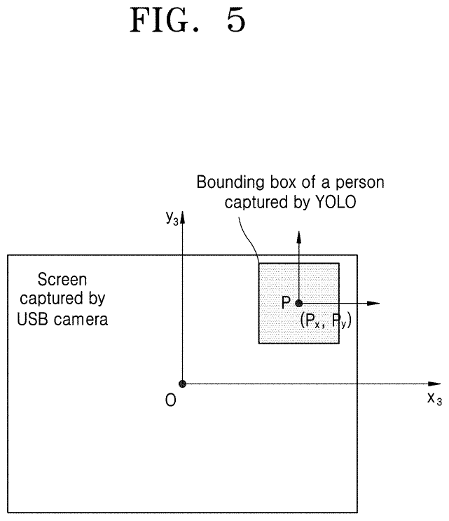

FIG. 5 is a diagram illustrating a state in which an end effector according to an embodiment of the present disclosure is aligned with a bounding box of a person.

FIG. 5 is a diagram illustrating a shape in which an end effector according to an embodiment of the present disclosure is aligned in a person's bounding box.

Referring to FIG. 5, when a person is detected, coordinates of the person's bounding box may be published by a YOLO algorithm, a custom program may calculate center coordinates of the bounding box there, and through this coordinate information, an actuator command for the manipulator 200 may be generated.

The manipulator 200 may include three actuators. One actuator may be used to perform a yaw motion, and two actuators may be used to perform a pitch motion to form the manipulator 200 with 3 degrees of freedom (DOF).

The YOLO algorithm may operate by aligning the center of the USB camera 310 to the center of the bounding box of the YOLO algorithm. As illustrated in FIG. 5, a point O may be aligned with a point P. In this case, the point O may change according to the manipulator 200, and the point P may change according to movement of a person.

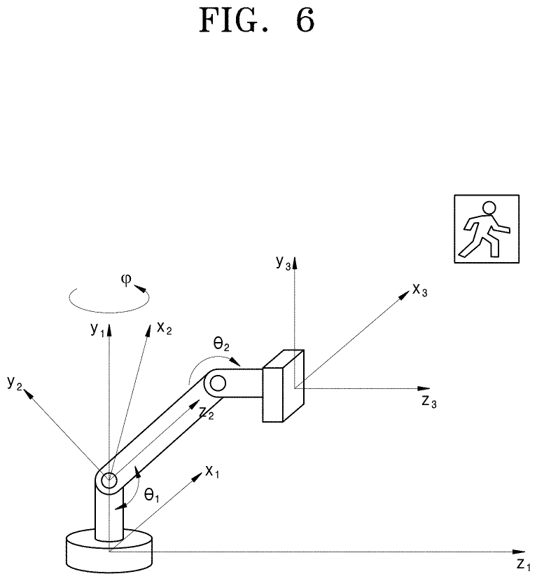

Coordinate axes of the manipulator 200 may be visualized as illustrated in FIG. 6. FIG. 6 is a view illustrating coordinate axes of a manipulator according to an embodiment of the present disclosure.

According to the present embodiment, a YOLO algorithm may be used to obtain coordinate values of a face and obtain temperature values of corresponding coordinate points. Through this, when an object with a higher temperature than a person's face is detected on a screen other than the person's face, a possibility of an error in which a high temperature may be detected may be blocked even though a person has a normal body temperature, and temperature values are accurately acquired.

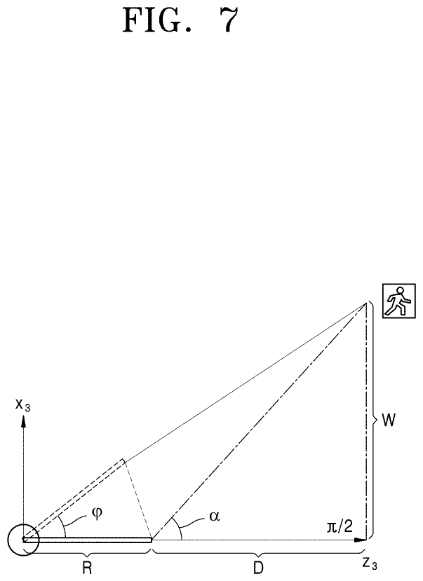

FIG. 7 illustrates how yaw is calculated. Referring to FIG. 7,

$$tan ( α ) = \frac{W}{D} and tan ( φ ) = \frac{W}{R + D} .$$

Because R=˜0.1 and D=2 to 3 m, R+D may be approximated to D. Therefore, it may be tan(α)≈tan(φ), α≈φ. The largest viewing angle of a camera may be 60 degrees horizontally and vertically, and each image may have a resolution of 640×480 pixels.

$${P}_{{x}_{m a x}} = 320 , {α}_{m a x} = \frac{π}{6}$$

In addition,

$$\frac{α}{{α}_{m a x}} = \frac{{P}_{x}}{{P}_{{x}_{m a x}}}$$

Therefore, yaw may be calculated to be

$$φ \approx α = \frac{π}{6 * 320} * {P}_{x} .$$

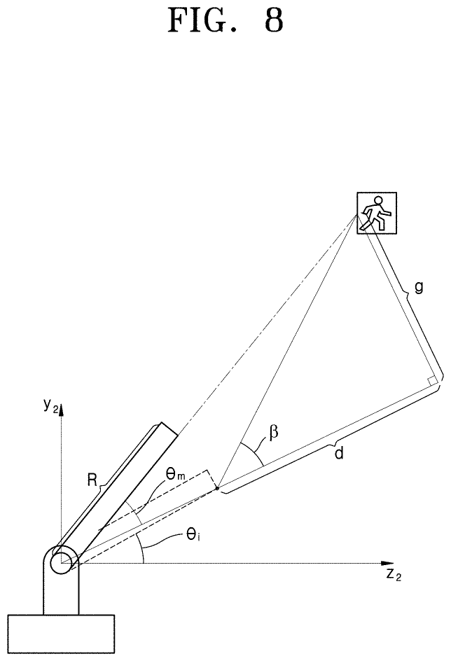

FIG. 8 illustrates how a pitch is calculated. Referring to FIG. 8, the manipulator 200 may have three DOFs, but the end effector 300 has only one overall angle aligned with a person's forehead, and thus, this may be effectively simplified to a pan-tilt system.

$$tan ( β ) = \frac{g}{d} and tan ( {θ}_{m} ) = \frac{g}{R + d} .$$

In addition, d is significantly greater than R, and thus, R+d may approximate d.

Therefore,

$$tan ( {θ}_{m} ) = \frac{g}{R + d} \approx \frac{g}{d} = tan ( β ) .$$

In addition,

$$\frac{β}{{β}_{m a x}} = \frac{{P}_{y}}{{P}_{{y}_{m a x}}} .$$

Therefore, a pitch may be calculated to be

$${θ}_{m} \approx β = \frac{π}{6 * 240} * {P}_{y} .$$

Coordinate information of a bounding box obtained from the YOLO algorithm may be sent to a smartphone through a web server. The Android application of the smartphone may extract coordinate information through a web crawler and the information may be used for temperature measurement.

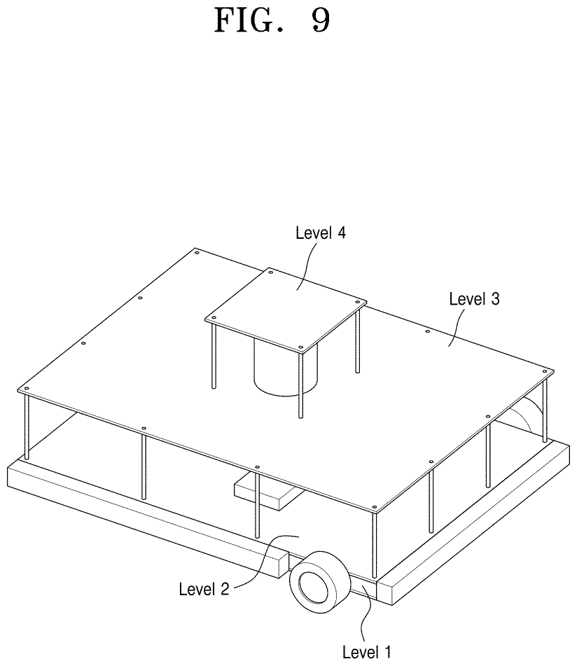

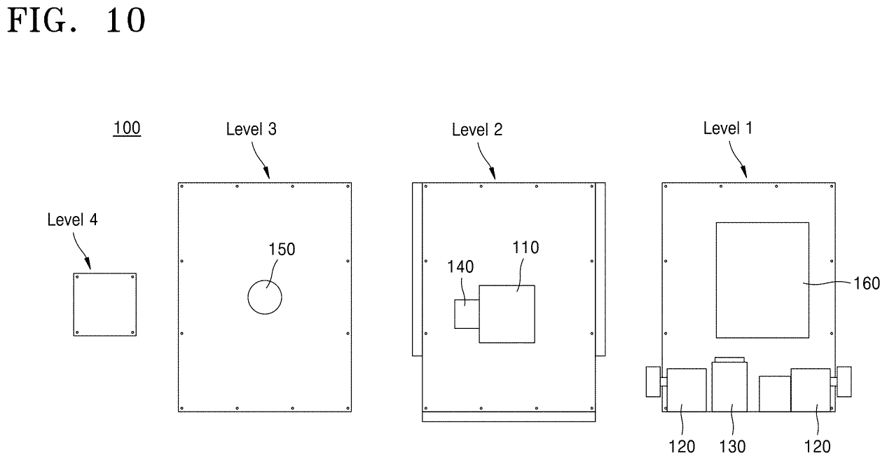

FIG. 9 is a view illustrating a shape of a mobile robot according to an embodiment of the present disclosure. FIG. 10 is a view illustrating internal configurations of the mobile robot according to an embodiment of the present disclosure.

Referring to FIGS. 9 and 10, the mobile robot 100 may stably support the manipulator 200 and perform simultaneous localization and mapping (SLAM), autonomous search, and path planning. With the SLAM, the mobile robot 100 may acquire a map through mapping and find out a location within the map (localization) at the same time. Navigation is moving from one place to another place and may be dependent upon localization, path planning, and mapping.

The mobile robot 100 according to an embodiment of the present disclosure may include a PC 110 that controls movement of the mobile robot 100, a plurality of actuators 120 that are highly compatible with an ROS and generate power through a provided motor, a first board 130 for control of the plurality of actuators and communication between the PC 110 and the plurality of actuators 120, a second board 140 for communication between the plurality of actuators of the manipulator 200 and the PC 110, a lidar system 150 used for remote sensing for SLAM, path planning, and autonomous search, and a battery 160 that provides a large capacity and necessary currents and voltages and simultaneously powers the PC 110 and the manipulator 200 without encountering overcurrent issues.

The lidar system 150 may remotely sense a person around an autonomously navigating robot. The manipulator 200 may be installed above the lidar system 150.

When the manipulator 200 is placed at the same level as the lidar system 150, the lidar system 150 may erroneously interpret the manipulator 200 as an obstacle, and thus, an error may occur in path analysis of the lidar system 150. Accordingly, as in the present embodiment, by installing the manipulator 200 above the lidar system 150, remote sensing of the lidar system 150 may be normally performed, and at the same time, an operation of the manipulator 200 may move more naturally at a position spaced apart from components of the mobile robot 100. Remote sensing information sensed by the lidar system 150 may be sent to the PC 110, and the PC 110 may move the mobile robot 100 to a place where there is a person, based on the sent information.

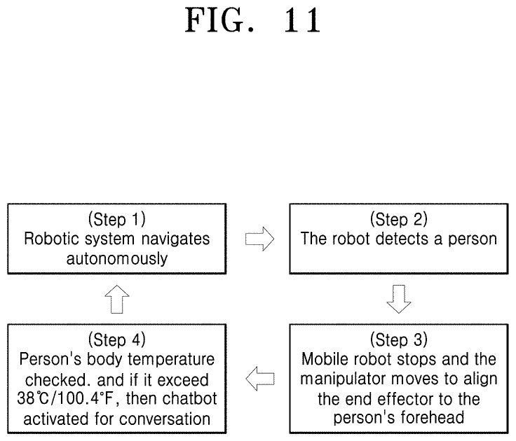

FIG. 11 is a flowchart illustrating an autonomously navigating robot operating system according to an embodiment of the present disclosure.

Referring to FIG. 11, the autonomously navigating robot operating system according to an embodiment of the present disclosure includes a step in which an autonomously navigating robot performs autonomous navigation (Step 1), a step in which the autonomously navigating robot detects a person (Step 2), a step in which a person's body temperature is checked, and when the body temperature exceeds a standard fever temperature, a chatbot is activated to converse with the person and check the person for symptoms of COVID-19 (Step 3).

An autonomously navigating robot according to an embodiment of the present disclosure may measure a person's body temperature while repeatedly performing the steps described above and may safely check whether or not the person is infected with COVID-19 through conversation.

As described above, the present disclosure is described with reference to the embodiments illustrated in the drawings, but these are only examples. Those skilled in the art may fully understand that various modifications and equivalent other embodiments may be possible from the embodiments. Therefore, the true technical protection scope of the present disclosure should be determined based on the appended claims.

The specific technical contents described in the embodiments are examples and do not limit the technical scope of the embodiments. In order to concisely and clearly describe the present disclosure, descriptions of general techniques and configurations of the related art may be omitted.

In addition, connections or connection members of lines between the components illustrated in the drawings illustratively show functional connections and/or physical or circuit connections and may be represented by a variety of additional functional connections, physical connections, or circuit connections that are replaceable or additional in an actual device. In addition, unless there is a specific reference to a member such as “essential” or “importantly”, the member may not be an essential component for the application of the present disclosure.

In the specification of the disclosure and in the claims, “the” or a word similar thereto may refer to both the singular and the plural unless otherwise specified. In addition, when a range is described in the embodiment, the range includes the disclosure to which individual values belonging to the range are applied (when there is no description to the contrary) and refers to each individual value constituting the range in the specification of the disclosure.

In addition, steps may be performed in an appropriate order unless the order is explicitly stated or there is no description to the contrary with respect to the steps constituting the method according to the embodiment. The embodiments are not limited to the order of description of the steps.

All examples or example terminology (for example, and so on and or so on) in the embodiment are merely for describing the embodiment in detail, and unless limited by the claims, the scope of the embodiment is not limited by the examples or example terminology. In addition, those skilled in the art will recognize that various modifications, combinations, and changes may be made depending on design conditions and factors within the scope of the appended claims or their equivalents.

In an autonomously navigating robot and an operation system thereof according to an embodiment of the present disclosure, the robot navigates autonomously, and thus, persons do not need to wait in long lines for a COVID-19 test, and a manipulator operation system allows the autonomously navigating robot to automatically adjust a thermal camera to measure a temperature without need for a person to move and align his/her face to a screen.

In addition, an autonomously navigating robot and an operation system thereof according to an embodiment of the present disclosure may increase measurement accuracy of a body temperature, thereby increasing accuracy of a COVID-19 test, and this is because, when measuring a body temperature at indoor and outdoor entrances in cold winter, there is a probability that a subject's body temperature may be measured to be less than an actual temperature due to influence of a low external temperature, whereas the autonomously navigating robot and the operation system may repeatedly measure a body temperature indoors.

In addition, a person measuring the temperature does not measure a temperature directly, and an autonomously navigating robot measures the temperature, and thus, the person measuring the temperature may be prevented from being infected with the virus.

Effects of the present disclosure are not limited to the effects described above, and other effects not described will be clearly understood by those skilled in the art from the description of claims.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. An autonomously navigating robot comprising:
an end effector configured to measure a body temperature of a person and, in response to the body temperature exceeding a standard fever temperature, activate a chatbot to check symptoms of Covid-19;
a manipulator configured to align the end effector with a forehead of the person; and
a mobile robot configured to detect the person and move the end effector and the manipulator to a position where the person is located by performing autonomous navigation.

2. The autonomously navigating robot of claim 1, wherein the mobile robot includes a personal computer configured to control a movement of the mobile robot.

3. The autonomously navigating robot of claim 1, wherein the mobile robot includes a lidar system configured to remotely sense the person by performing simultaneous localization and mapping (SLAM), autonomous search, and path planning.

4. The autonomously navigating robot of claim 3, wherein the manipulator is installed above the lidar system.

5. The autonomously navigating robot of claim 1, wherein the manipulator includes three actuators.

6. The autonomously navigating robot of claim 5, wherein one of the three actuators is configured to perform a yaw motion, and the other two actuators are configured to perform a pitch motion.

7. The autonomously navigating robot of claim 1, wherein the end effector includes a Universal Serial Bus camera configured to provide a real-time image.

8. The autonomously navigating robot of claim 7, wherein:
the Universal Serial Bus camera is configured to transmit the real-time image to a personal computer of the mobile robot, and
a you-only-look-once algorithm executed by the personal computer is configured to detect the person.

9. The autonomously navigating robot of claim 8, wherein, in response to the person being detected, the you-only-look-once algorithm is configured to calculate coordinates of a bounding box of a face of the person and a center coordinate of the bounding box and generate an actuator command for the manipulator based on information of the coordinates.

10. The autonomously navigating robot of claim 9, wherein the you-only-look-once algorithm is configured to acquire a coordinate value of the face of the person and acquire a temperature value of a point of the coordinate value.

11. The autonomously navigating robot of claim 7, wherein the end effector further includes a thermal camera configured to determine the body temperature.

12. The autonomously navigating robot of claim 11, wherein one end of a fixed portion and one end of a thermal camera fixed hanger are arranged between the thermal camera and the Universal Serial Bus camera.

13. The autonomously navigating robot of claim 1, wherein the end effector includes:
a fixed portion configured to form a skeleton of the end effector; and
a thermal camera fixed mechanism configured to fix the thermal camera arranged in the end effector.

14. The autonomously navigating robot of claim 13, wherein a Universal Serial Bus camera, the thermal imager, and a phone case are mounted on the fixed portion.

15. The autonomously navigating robot of claim 1, wherein the end effector is equipped with a smartphone configured to provide a user interface.

16. The autonomously navigating robot of claim 15, wherein an Android custom application of the smartphone is configured to display, on a thermal image provided by the thermal camera, a point with a highest temperature among nine temperature points including one temperature point of provided coordinates and eight temperature points around the provided coordinates.

17. The autonomously navigating robot of claim 16, wherein, in response to a temperature exceeding a fever threshold value among the nine temperature points being detected, a screen of the smart phone is configured to be changed to a help screen, and a natural language understanding artificial intelligence chatbot is configured to be activated.

18. The autonomously navigating robot of claim 17, wherein:
the natural language understanding artificial intelligence chatbot is configured to converse with a user about vaccinations and potential symptoms of Covid-19, and
in response to a predefined set of intents and 10 to 15 sample phrases being provided, a chatbot framework is configured to respond by accurately extracting a meaning of each of the sample phrases and self-learning to match the intents.

19. An autonomously navigating robot operation system comprising:
a memory storing instructions; and
a processor configured to execute the instructions to:
perform autonomous navigation by using an autonomously navigating robot;
detect a person by using the autonomously navigating robot;
align an end effector with a forehead of the person by stopping the autonomously navigating robot and moving a manipulator; and
check a body temperature of the person and, in response to the body temperature exceeding a standard fever temperature, activate a chatbot to converse with the person and check the person for symptoms of Covid-19.

20. A non-transitory computer readable recording medium storing instructions, when executed by one or more processors, to perform a method of operating an autonomously navigating robot, the method comprising:
performing autonomous navigation by using an autonomously navigating robot;
detecting a person by using the autonomously navigating robot;
aligning an end effector with a forehead of the person by stopping the autonomously navigating robot and moving a manipulator; and
checking a body temperature of the person and, when the body temperature exceeds a standard fever temperature, activating a chatbot to converse with the person and check the person for symptoms of Covid-19.
